(19) United States
(12) Patent Application Publication
(10) Pub. No.: US 2019/0336675 A1
(43) Pub. Date: Nov. 7, 2019

(54) FLOW CONTROLLER FOR VESSEL BANDING

(71) Applicant: Renal Devices Inc., Amherst, NY (US)

(72) Inventors: Mark D. Lauren, Amherst, NY (US); Daniel J. D'Arcy, Orchard Park, NY (US)

(21) Appl. No.: 16/391,449

(22) Filed: Apr. 23, 2019

Related U.S. Application Data
(60) Provisional application No. 62/762,465, filed on May 7, 2018.

(51) Int. Cl. A61M 1/36; A61M 39/28; A61M 39/10

(52) U.S. Cl. CPC A61M 1/3655; A61M 2039/1044; A61M 39/10; A61M 39/288

(57) ABSTRACT

A flow controller arranged to provide controlled constriction of a hollow body conduit, including a catheter including a first end and a second end, a strap slidably arranged within the catheter, the strap including a third end and a fourth end, the third end arranged to be connected to the catheter to form a loop, and a tensioning means connected to the second end and the fourth end, wherein the tensioning means is operatively arranged to axially displace the strap relative to the catheter to increase and decrease a diameter of the loop.

### CROSS-REFERENCE TO RELATED APPLICATIONS

This application claims the benefit under 35 U.S.C. § 119(e) of U.S. Provisional Application No. 62/762,465, filed May 7, 2018, which application is incorporated herein by reference in its entirety.

### FIELD

The present disclosure relates to the field of arteriovenous fistulas, and more particularly, to a device that controls the flow rate of an arteriovenous fistula.

### BACKGROUND

Arteriovenous (AV) fistulas remain the preferred modality for providing blood access for hemodialysis. The surgical creation of an AV fistula involves connecting a long vein segment to an arterial source. The vein dilates from the increased blood pressure and begins to remodel on a cellular level. The vein is then left to “mature” for about one to three months. To be successful, the mature fistula needs a blood flow of about 600 milliliters/minute, be 5-6 mm in diameter, and be able to be cannulated three times per week for dialysis access. Vascular grafts may also serve as an AV fistula.

Some fistulas can reach pathologically high flows, or high flow rates, for example, 2-3 liters/minute, for a variety of reasons. Fistulas (particularly vascular grafts) that are fed by the brachial artery are most susceptible. Flow reduction methods generally reduce fistula flow from over 2 liters/minute to about 1 liter/minute.

An AV fistula can be a 6 mm diameter vessel flowing directly from a medium size artery into a medium size vein. The fistula generally becomes the preferred route for most of the feeding arterial blood rather than the higher resistance distal arteries. Retrograde flow in the distal artery, physiologic steal, is common and generally asymptomatic, stealing some blood from the hand. In some patients, high-flow fistulas create serious ischemia. Ischemic symptoms range from tingling, numbness, and extreme cold, to rest pain and tissue necrosis. Dialysis access associated ischemia (DAAS) is the primary rationale for intervention to reduce fistula flow. While the incidence of DAAS is low, the morbidity can be significant. Flow reduction provides near immediate symptom relief.

The creation of an AV fistula also has immediate and universal negative cardiac effects including an increase in cardiac output (CO), heart rate, and stroke volume. If fistula flow increases to over about 1.5 liters/minute, the decreased peripheral resistance, increased right atrial and pulmonary artery pressure, and increased left ventricular end-diastolic pressure causes the myocardium to decompensate. At that point, the patient has symptoms of heart failure. Reducing AV fistula flow (with increased peripheral resistance) can provide near immediate cardiac benefits.

Current methods to reduce fistula flow include vessel surgeries and banding procedures. In practice, each surgeon typically has a preferred flow reduction technique—generally following how one is trained. Vessel surgeries reroute some of the arterial inlet blood to the fistula, thereby reducing fistula flow and increasing distal perfusion. The most common surgeries are distal revascularization with interval ligation (DRIL) and revision using distal inflow (RUDI).

Surgical procedures provide only approximate control of blood flow, based on surgical experience. Requiring general sedation, the amount of flow reduction is considered appropriate, just based on observations of the return of color and a radial pulse. These procedures do not allow blood flow to be individually titrated for each patient. Surgeries are also significant procedures for most patients, being expensive and having associated morbidity.

A primary physiologic deficiency of surgical flow reduction methods is that it does not increase peripheral resistance. As a result, surgically-based flow reductions do not provide any cardiac benefit. Since vessel surgeries do not restrict the fistula itself, their only specific advantage is a slightly lower thrombosis rate compared with surgical banding.

Surgical banding reduces flow by directly constricting the fistula. Banding has the important physiologic advantage (vs. vessel surgeries) of increasing peripheral resistance. Fistula banding therefore provides relief from both cardiac and ischemic symptoms.

With surgical plication (surgical banding), the anastomosis region of the fistula is narrowed using a row of stitches to form a pleat. This is a poorly-controlled procedure which is frequently performed two or three times on the same individual. Plication has shown variable clinical results—with a wide range of fistula thrombosis rates. While not requiring general sedation, plication is irreversible, poorly-controlled, and has thrombosis possibilities.

Clinical reports also describe using a single loop of suture to serve as a flow reduction band around a fistula. The term “suture-banding” is used to distinguish this method from plication-type surgical banding. These reports represent the most similar methodologies to the current invention.

The use of a suture was first reported in 2006. See N. Goel, et al., Minimally Invasive Limited Ligation Endoluminal-assisted Revision (MILLER) for Treatment of Dialysis Access-associated Steal Syndrome, 70 KIDNEY INT'L 765 (2006). This procedure inserts an intraluminal balloon into the fistula, which is used as a sizing dowel. A loop of 2-0 suture is then wrapped around the fistula and tightly snugged to the balloon. The balloon is then deflated and removed. Final flow values are determined solely by the chosen balloon diameter and not clinical symptoms. There is no way to exercise fine control over the flow. The need to apply a second band is common. An alternative method places a coronary dilator outside the fistula and tightens a suture around the fistula and dilator. See Gregg A. Miller, et al., The MILLER Banding Procedure is an Effective Method for Treating Dialysis-associated Steal Syndrome, 77 KIDNEY INT'L 359 (2010). The dilator is then removed. This method avoids inserting a balloon, but significantly stresses the fistula.

The primary drawbacks of suture-banding are the approximate nature of its flow reduction and the high wall stress induced by a single wrap of suture. Flow reduction is dictated by the semi-subjective selection of balloon diameter. The method also has the potential for thrombosis of the fistula. However, acceptable thrombosis rates have been reported by different groups using suture-based methods. See Pratik A. Shukla, et al., The MILLER Banding Procedure as a Treatment Alternative for Dialysis Access Steal Syndrome: A Single Institutional Experience, 40 CLINICAL IMAGING 569 (2016).

A number of vessel flow reduction devices have been reported in the patent literature. Some are applied to vessels in general, and some are specific to AV fistula. The AV fistula devices are mainly designed to reduce fistula flow between dialysis sessions when high flow is not needed. Many are elaborate and inherently impractical for a commercial medical device.

The universal shortcoming of all current surgical and banding flow reduction methods is the inability to individually titrate the flow to clinical symptoms. There is a need for a convenient atraumatic, controllable method to band a vessel for the purposes of providing controlled flow reduction.

Thus, there is a long felt need for an implantable device that provides controlled restriction of a blood conduit.

### SUMMARY

According to aspects illustrated herein, there is provided a flow controller arranged to provide controlled constriction of a hollow body conduit, comprising a catheter including a first end and a second end, a strap slidably arranged within the catheter, the strap including a third end and a fourth end, the third end arranged to be connected to the catheter to form a loop, and a tensioning means connected to the second end and the fourth end, wherein the tensioning means is operatively arranged to axially displace the strap relative to the catheter to increase and decrease a diameter of the loop.

According to aspects illustrated herein, there is provided a flow controller arranged to provide controlled constriction of a hollow body conduit, comprising a catheter including a first end and a second end, a strap slidably arranged within the catheter, the strap including a third end and a fourth end, the third end operatively arranged to be removably connected to the catheter to form a loop, wherein the loop is arranged around the hollow body conduit, and a tensioning means, including a housing connected to the second end, and a threaded rod slidably arranged in the housing, wherein the threaded rod is connected to the fourth end and is axially displaced relative to the housing to increase and decrease a diameter of the loop.

According to aspects illustrated herein, there is provided a flow controller device designed to overcome the recognized deficiencies of prior art fistula flow control methods. The device provides an atraumatic band that may be placed around the fistula. The diameter of the band may be finely controlled using an integrated and disposable controller or tensioning means. Band diameter, and thereby fistula flow, may be changed in response to observed clinical symptoms and measurements. When finished, a stainless-steel ring is pinched to fix the band diameter and the controller or tensioning means is removed, leaving only the vessel band in place.

The device comprises an adjustable strap (or band) that may be installed by leading it around a blood conduit. The strap itself typically has a cylindrical cross-section. The strap material may be a bead made of a flexible polymer such as TEFLON® polytetrafluoroethylene (PTFE). The diameter of the bead material or strap may be approximately 1.5 mm. The loop formed by the band may have a maximum inside diameter of approximately 8-12 mm and a minimum inside diameter of 3-5 mm.

The strap is considered to have a free end and a moveable end. The free end is fed around the vessel and attached to a fixed collar component or connector. Once secured to the collar or connector, the strap forms a ring or loop, and is in its fully untightened position (i.e., having a maximum diameter). There is generally a separate component or connector attached to the free end of the strap (strap tail) to facilitate proper attachment of the strap to the collar. The moveable end of the strap may pass through a catheter that is connected to a central hole in the collar component or connector.

The catheter and band may lead from the collar component to a controller or tensioning means. The controller or tensioning means provides fine and controlled movement of the strap inside the catheter to effect controlled changes in the loop diameter. This allows the loop diameter to be changed to reflect clinical symptoms for an individual patient.

In some embodiments, the controller or tensioning means uses a threaded element that is fitted with an anti-rotation means to provide linear motion when activated by a nut. The strap may be attached to the threaded element which then provides controlled movement of the strap.

When flow control is complete, a short stainless-steel sleeve is cinched over the catheter to lock the strap to the catheter, thereby fixing the loop diameter. The catheter and strap may then be cut and the controller or tensioning means removed, leaving the strap in place.

The controller or tensioning means may include the means to indicate the diameter of the strap loop. In some embodiments, the controller or tensioning means comprises transparent or translucent material which allows the internal screw to be visualized and used to indicate loop diameter. The controller or tensioning means may also indicate the rotation direction for opening and closing the strap (i.e., which circumferential direction tightens and untightens the loop). The controller or tensioning means may be fitted with a means to limit the displacement of the threaded rod in both axial directions.

These and other objects, features, and advantages of the present disclosure will become readily apparent upon a review of the following detailed description of the disclosure, in view of the drawings and appended claims.

### BRIEF DESCRIPTION OF THE DRAWINGS

Various embodiments are disclosed, by way of example only, with reference to the accompanying schematic drawings in which corresponding reference symbols indicate corresponding parts, in which:

FIG. 1A is a perspective view of a flow controller in an unsecured state;

FIG. 1B is a perspective view of the flow controller shown in FIG. 1A, in a secured state;

FIG. 2 is an exploded view of the flow controller shown in FIG. 1B;

FIG. 3 is a cross-sectional view of the flow controller taken generally along line 3-3 in FIG. 1B;

FIG. 4 is a perspective view of the flow controller shown in FIG. 1B, in a tightened state;

FIG. 5 is a perspective view of the securing means shown in FIG. 4;

FIG. 6 is a perspective view of the housing shown in FIG. 1A; and,

FIG. 7 is a perspective view of the threaded rod shown in FIG. 2.

### DETAILED DESCRIPTION

At the outset, it should be appreciated that like drawing numbers on different drawing views identify identical, or functionally similar, structural elements. It is to be understood that the claims are not limited to the disclosed aspects.

Furthermore, it is understood that this disclosure is not limited to the particular methodology, materials and modifications described and as such may, of course, vary. It is also understood that the terminology used herein is for the purpose of describing particular aspects only, and is not intended to limit the scope of the claims.

Unless defined otherwise, all technical and scientific terms used herein have the same meaning as commonly understood to one of ordinary skill in the art to which this disclosure pertains. It should be understood that any methods, devices or materials similar or equivalent to those described herein can be used in the practice or testing of the example embodiments. The assembly of the present disclosure could be driven by hydraulics, electronics, pneumatics, and/or springs.

It should be appreciated that the term “substantially” is synonymous with terms such as “nearly,” “very nearly,” “about,” “approximately,” “around,” “bordering on,” “close to,” “essentially,” “in the neighborhood of,” “in the vicinity of,” etc., and such terms may be used interchangeably as appearing in the specification and claims. It should be appreciated that the term “proximate” is synonymous with terms such as “nearby,” “close,” “adjacent,” “neighboring,” “immediate,” “adjoining,” etc., and such terms may be used interchangeably as appearing in the specification and claims.

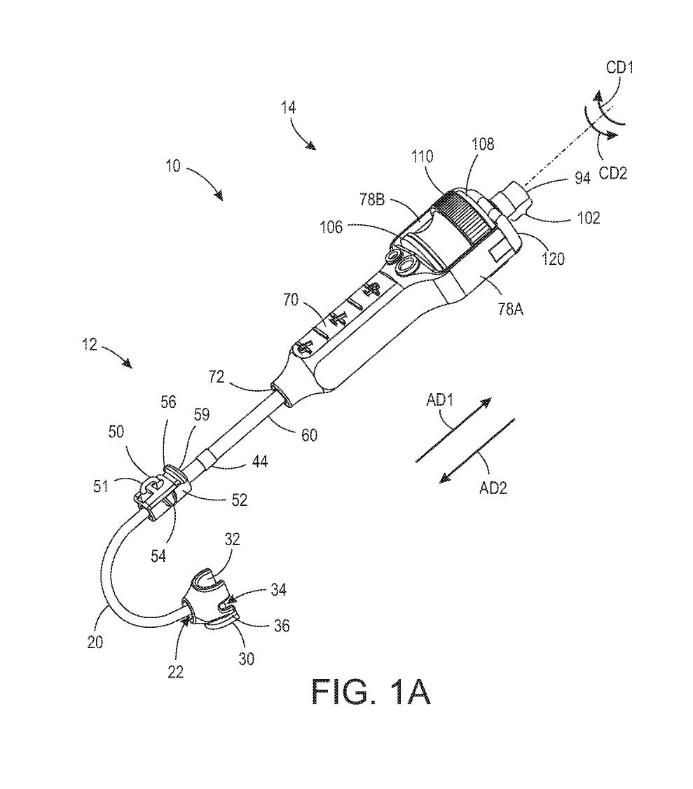

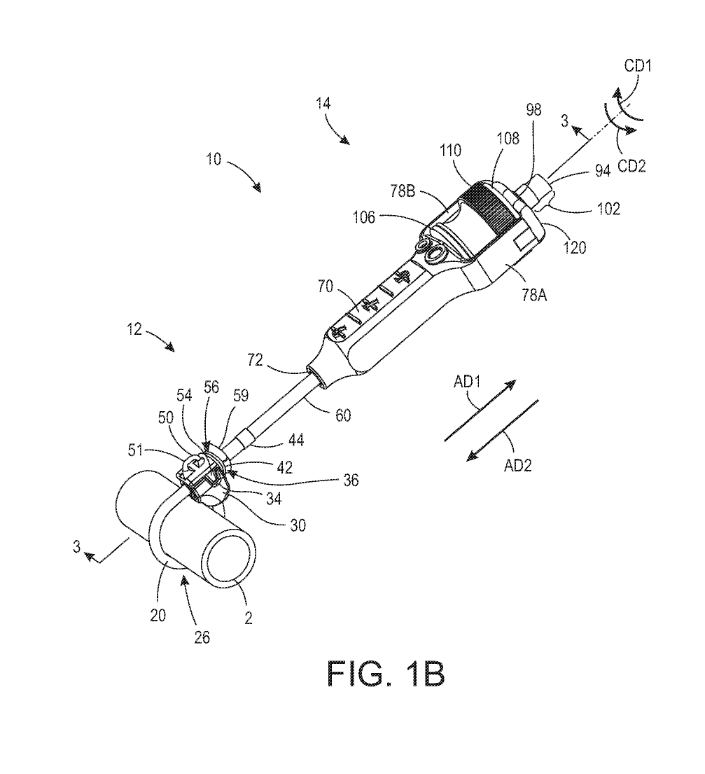

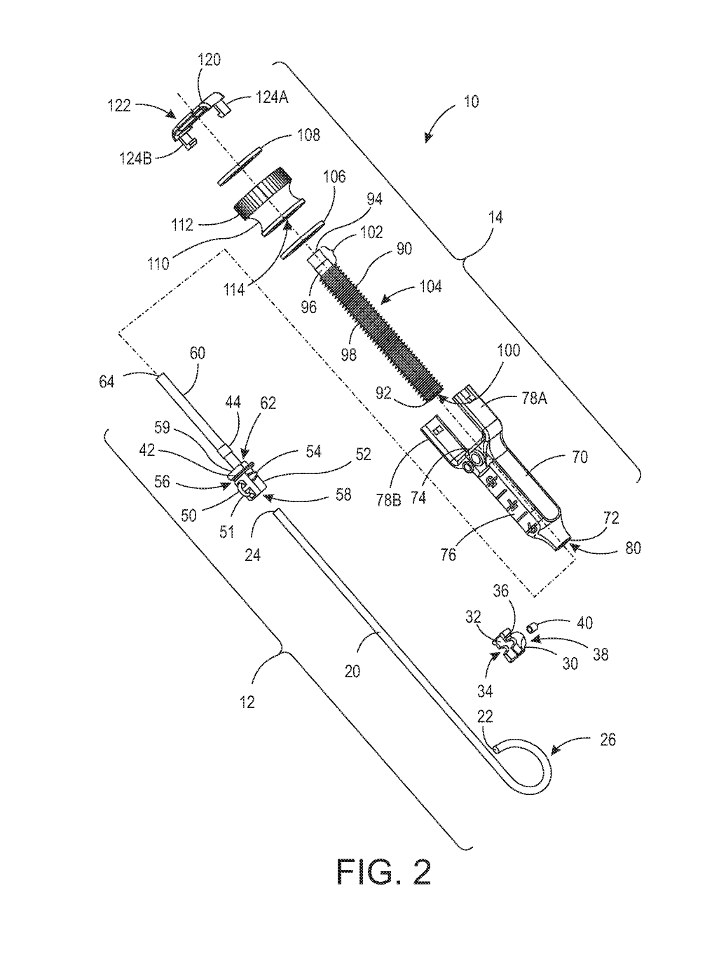

Referring now to the figures, FIG. 1A is a perspective view of flow controller 10 in an unsecured state. FIG. 1B is a perspective view of flow controller 10 in a secured state. FIG. 2 is an exploded view of flow controller 10. FIG. 3 is a cross-sectional view of flow controller 10 taken generally along line 3-3 in FIG. 1B. Flow controller 10 generally comprises securing means 12 and tensioning means 14. The following description should be read in view of FIGS. 1A-3.

Securing means 12 comprises strap 20, female connector 30, male connector 50, and catheter 60. Securing means 12 may further comprise suture 42 and/or tube 44.

Strap 20 generally comprises a flexible material and includes end 22 and end 24. In some embodiments, strap 20 comprises a cylindrical cross-section having a diameter of approximately 1.5-3 mm. In some embodiments, strap 20 comprises a cylindrical cross-section having a diameter of approximately 1-5 mm. It should be appreciated that strap 20 may comprise any geometry cross-section (e.g., ovular, rectangular, etc.) suitable for forming a loop and tightening around a vessel (e.g., vein, gastrointestinal (GI) tract, urethra, genitourinary, vascular graft, blood vessel, artery, lungs, etc.). In some embodiments, strap 20 comprises TEFLON® PTFE, or another biocompatible polymer. In some embodiments, end 22 of strap 20 is etched to provide a surface for bonding or fixing to female component 30.

Female connector 30 is generally partially tubular in shape and comprises radially inward facing surface 32, notches 34, annular groove 36, and hole 38. End 22 is operatively arranged to engage hole 38 to fixedly secure strap 20 to female connector 30. Female connector 30 may further comprise bushing 40 to aid in fixedly securing end 22 in hole 38. For example, bushing 40 may be bonded to etched end 22 using a heat-cured epoxy, and bushing 40 is then bonded within hole 38. In some embodiments, bushing 40 comprises stainless steel. Bushing 40 may further act as a radiopaque marker. Female connector 30 is operatively arranged to connect to male connector 50 such that strap 20 forms loop 26, as will be discussed in greater detail below. In some embodiments, female connector 30 comprises PEBAX® polyether block amide (PEBA). Female connector 30 may also form part of the vein or vessel contact surface, as shown in FIG. 1B. As such, female connector 30 comprises a curvature, arranged to contact vein 2, comprising a diameter that is proximate to the most common final diameter of loop 26 for a particular application.

Male connector 50 is generally tubular and comprises radially outward facing surface 52, protrusions 54, annular groove 56, through-bore 58, and surface 59. Protrusions 54 are arranged on radially outward facing surface 52. Radially outward facing surface 52 is operatively arranged to engage radially inward facing surface 32 to connect male connector 50 and female connector 30. Protrusions 54 are operatively arranged to engage notches 34 to aid in the proper connection of female connector 30 to male connector 50 and to properly align annular groove 36 with annular groove 56. The diameter of radially inward facing surface 32 may be of a certain dimension such that it “snaps” together with radially outward facing surface 52, such that female connector 30 is removably secured to male connector 50. When female connector 30 is connected to male connector 50, annular groove 36 is aligned with annular groove 56. Suture 42 may be secured in annular grooves 36 and 56 to fixedly secure female connector 30 to male connector 50. Suture 42 may comprise any material suitable to hold female connector 30 to male connector 50. Through-bore 58 extends through male connector 50 and is arranged to be slidably engaged with strap 20 and fixedly secured with catheter 60, as will be discussed in greater detail below. The arrangement of female connector 30 and male connector 50 provides a means for connecting end 22 of strap 20 to catheter 60 to form loop 26 that helps maintain a circular loop geometry as the diameter of loop 26 changes and prevents pinching. As shown in FIG. 2, female connector 30, which is connected to end 22 of strap 20, is arranged around vein or vessel 2 and connected to male connector 50. In some embodiments, male connector 50 comprises PEBAX® PEBA. Male connector 50 may further comprise attachment 51 for securing male connector 50 to the surrounding tissue to prevent securing means 12 from migrating. Attachment 51 may be formed as a loop on the top surface of male connector 50, through which a suture can be connected to surrounding tissue. Attachment 51 also provides a convenient means to hold male connector 50 while snapping female connector 30 thereto. In an example embodiment, end 22 of strap 20 directly connects to male component 50 without the need for female component 30, for example, by using a metal strap. It should be appreciated that connector 50 may be a female connector and connector 30 may be a male connector, and that any suitable connection method may be used to connect end 22 to catheter 60.

Catheter 60 is generally a tube comprising end 62 and end 64. In some embodiments, catheter 60 is a tube that allows sliding engagement with strap 20. In some embodiments, catheter 60 is a collar that allows sliding engagement with strap 20 and replaces, for example, male collar 50. In some embodiments, catheter 60 is any annular connecting means suitable for sliding engagement with strap 20. In some embodiments, end 62 is arranged to be connected to male connector 50. In some embodiments, end 62 is fixedly secured to surface 59 and concentrically aligned with through-bore 58. In some embodiments, end 62 engages through-bore 58 and is fixedly secured therein. End 64 is arranged to be connected to housing 70. In some embodiments, end 64 is fixedly secured to end 72 and concentrically aligned with hole 80. In some embodiments, end 64 engages hole 80 and is fixedly secured therein. Strap 20 is slideably engaged within catheter 60. Specifically, end 24 of strap 20 is fed through male connector 50, catheter 60, and hole 80, housing 70, and threaded rod 90 and is connected to end 94 of threaded rod. It should be appreciated that strap 20 is slidable with respect to male connector 50, catheter 60, and housing 70. As such, as tensioning means 14 is tightened, thereby displacing threaded rod 90 with respect to housing 70, the diameter of loop 26 is reduced, as will be described in greater detail below. Catheter 60 may further comprise tube 44 slidably arranged thereon. When loop 26 is set at a sufficient diameter, tube 44 may be crimped in order to fixedly secure strap 20 and catheter 60 together, thereby locking in the diameter of loop 26. The locking function of tube 44 allows tensioning means 14 to be removed from securing means 12 while loop 26 remains secured at the set diameter around vein or vessel 2. In some embodiments, tube 44 comprises stainless steel. Tube 44 may further act as a radiopaque marker. In some embodiments, catheter 60 comprises PEBAX® PEBA. It should be appreciated, that in some embodiments, tensioning means 14, specifically housing 70 could be fixedly secured to male collar 50. In such embodiments, there is no need for catheter 60.

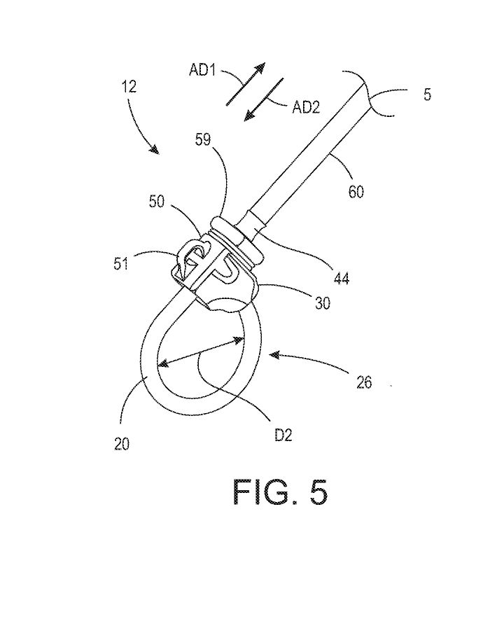

Housing 70 generally comprises end 72, end 74, measuring surface 76, and fork portions 78A-B. Housing 70 further comprises hole 80 proximate end 72, which terminates at surface 82, and hole 84, which extends from surface 82 to end 74 (best seen in FIG. 6). As previously discussed, end 64 of catheter 60 is arranged to be connected to housing 70 (e.g., at end 72 or in hole 80). It should be appreciated that, although housing 70 may be fixedly secured to end 72 of catheter 70, in some embodiments housing 70 may be removably engageable with end 72 of housing. For example, securing means 12 may be connected to a vein or vessel, after which time tensioning means 14 can be connected to securing means 12 in order to provide the necessary tightening of loop 26 around vein or vessel 2. End 24 of strap 20 is fed through hole 80 and is fixedly secured to threaded rod 90. Housing 70 further comprises protrusion 86 (see FIG. 6) which is arranged in hole 84. Fork portions 78A and 78B extend from end 74 and are operatively arranged to engage housing cap 120. In some embodiments, housing 70 comprises a transparent or translucent material, which allows the position of threaded rod 90 to be visualized. Measuring surface 76 may include calibrated measurements that indicate the diameter of loop 26, for example, in millimeters or inches. In some embodiments, measuring surface 76 comprises a scale, which is arranged to indicate the diameter of loop 26 based on the position of end 92 of threaded rod 90, or some other marking on threaded rod 90 (i.e., at a point between end 92 and end 94). In some embodiments, measuring surface 76 comprises a scale, which is arranged to indicate the linear displacement of strap 20, for example, in millimeters or inches. In some embodiments, measuring surface 76 comprises a small circle and a large circle which indicate in which circumferential direction nut 110 must be rotated in order to increase and decrease the diameter of loop 26. For example, according to FIG. 1B, rotating nut 110 toward the small circle (i.e., in circumferential direction CD1) decreases the diameter of loop 26, whereas rotating nut 110 toward the large circle (i.e., in circumferential direction CD2) increases the diameter of loop 26. In some embodiments, end 72 is spaced apart from surface 59 by approximately 2 cm.

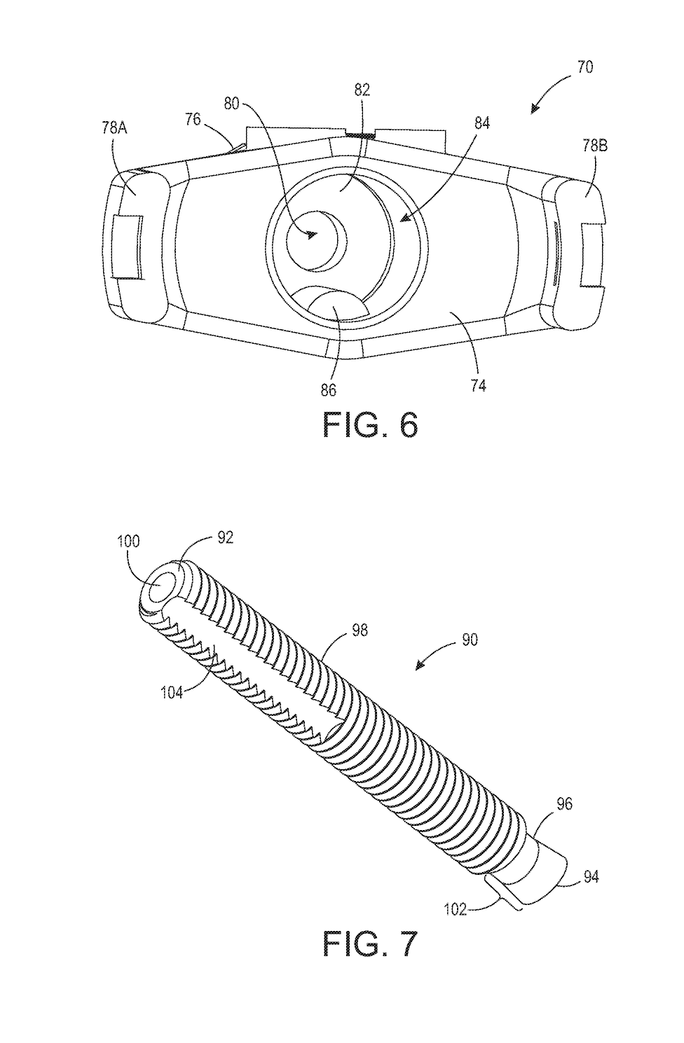

Threaded rod 90 generally comprises end 92, end 94, radially outward facing surface 96, threading 98, and through-bore 100. Threaded rod 90 is slidably arranged in hole 84. End 24 of strap 20 is fixedly secured to threaded rod 90 such that, when threaded rod 90 is displaced in axial direction AD1, end 22 is pulled in axial direction AD1 and the diameter of loop 26 decreases, thus further restricting the flow through vein 2. When threaded rod 90 is displaced in axial direction AD2, end 22 is pushed in axial direction AD2 and the diameter of loop 26 increases, thus reducing the restriction on the flow through vein 2. When flow controller 10 is in a fully untightened state, as shown in FIG. 3, end 92 of threaded rod 90 abuts against or is arranged substantially proximate to surface 82 of housing 70. As flow controller 10 is tightened, threaded rod 90 is displaced in axial direction AD1 thereby separating end 92 from surface 82. Threaded rod 90 is displaced relative to housing 70 via nut 110. Threaded rod 90 comprises crimp portion 102 arranged proximate end 94. As shown in the drawings, end 24 of strap 20 is fed through through-bore 100 and is fixedly secured at end 94 of threaded rod 90 via a crimp (i.e., crimp portion 102 is crimped or squeezed together around strap 20 and thus fixedly secures end 94 and end 24). It should be appreciated that any suitable method of fixedly securing strap 20 to threaded rod 90 may be used. It should also be appreciated that strap 20 need not be fed through through-bore 100 of threaded rod 90, but rather can be fixedly secured to end 92 or within through-bore 90 at a point between end 92 and 94. Threaded rod 90 further comprises groove 104 (see FIG. 7), which is arranged to engage protrusion 86 of housing 70 to prevent threaded rod from rotating relative to housing 70, within hole 84.

Nut 110 comprises grip 112 and hole 114. Nut 110 is axially arranged between housing cap 120 and housing 70, specifically end 74. Hole 114 comprises threading that engages with threading 98. Housing cap 120 is secured to housing 70. Specifically, clip portions 124A and 124B engage fork portions 78A and 78B. Clip portions 124A-B may comprise protrusions that engage slots in fork portions 78A-B. It should be appreciated that any suitable means for connecting housing cap 120 to housing 70 may be used, for example, adhesives, bolts, screws, rivets, soldering, welding, etc. With nut 110 engaged with threaded rod 90 and axially arranged between end 74 and housing cap 120, nut 110 can be rotated in circumferential direction CD1 or CD2 to tighten/loosen flow controller 10. For example, a user may use grip 112 to rotate nut 110 in circumferential direction CD1 to tighten loop 26 (i.e., displace threaded rod 90 in axial direction AD1) and circumferential direction CD2 to loosen loop 26 (i.e., displace threaded rod 90 in axial direction AD2). In some embodiments, a user may use grip 112 to rotate nut 110 in circumferential direction CD2 to tighten loop 26 (i.e., displace threaded rod 90 in axial direction AD1) and circumferential direction CD1 to loosen loop 26 (i.e., displace threaded rod 90 in axial direction AD2). In some embodiments, threaded rod 90 comprises a #10-32 screw. For a 32-pitch screw, four rotations of nut 110 corresponds to a 1 mm change in the diameter of loop 26. It should be appreciated that due to the engagement of nut 110 with threading 98, threaded rod 90 and thus strap 20 is prevented from axial displacement except when nut 110 is rotated, thus preventing unwanted loosening of loop 26.

Flow controller 10 may further comprise washers 106 and 108 arranged on opposite axial sides of nut 110 to aid in the reduction of friction between nut 110 and housing 70 and housing cap 120 during tightening and untightening (i.e., loosening). For example, washer 106 may be axially arranged between nut 110 and end 74 and washer 108 may be axially arranged between nut 110 and housing cap 120.

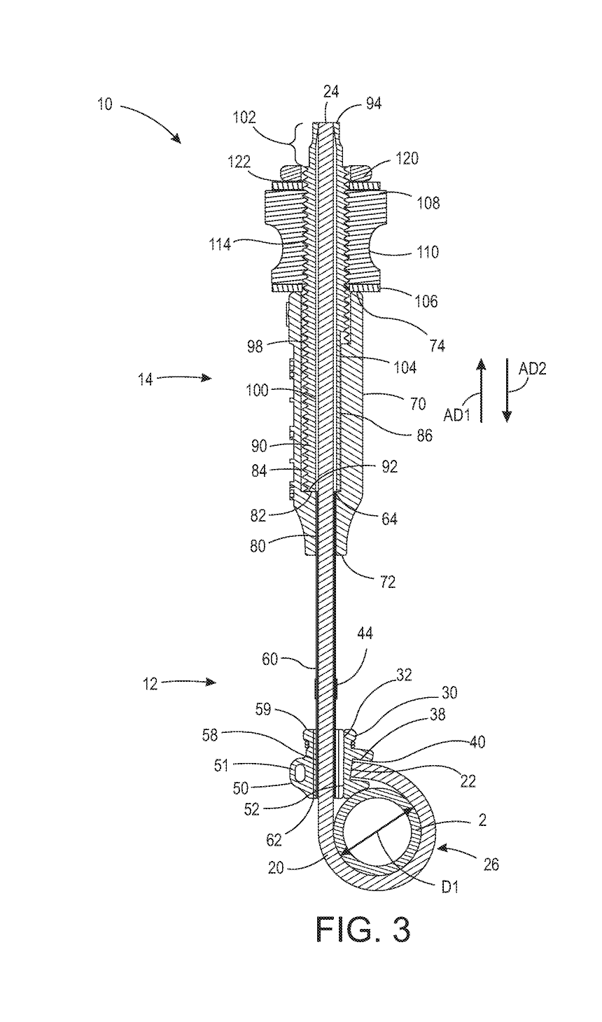

FIG. 4 is a perspective view of flow controller 10 in a tightened state. As shown in FIGS. 1B-3, loop 26 comprises diameter D1 in an untightened state. When flow controller 10 is in a fully untightened state, diameter D1 of loop 26 is at a maximum. As flow controller 10 is tightened, the diameter of loop 26 decreases. For example, in FIG. 4, flow controller 10 is in an at least partially tightened state and loop 26 comprises diameter D2, which is less than diameter D1. Specifically, flow controller 10 has been tightened such that threaded rod 90 has displaced length L. In some embodiments, the diameter of loop 26 ranges from a maximum of 20 mm (e.g., diameter D1) down to a minimum diameter of 5 mm. In some embodiments, the maximum inner diameter of loop 26 is 8-12 mm and the minimum inner diameter of loop 26 is 3-5 mm. Housing 70 may further comprise measuring surface 76 which allows a user to visually identify the magnitude of tension. For example, in a fully untightened state, as shown in FIGS. 1A-3, end 92 of threaded rod 90 is aligned with “9” on measuring surface 76. In FIG. 4, in an at least partially tightened state, end 92 is aligned with “6” on measuring surface 76, indicating that length L is equal to three (3) measuring units (e.g., 0.75 inches if each measuring unit is 0.25 inches, or 3 mm if each measuring unit is 1 mm). Once the desired diameter of loop 26 is set, tube 44 can be crimped to fixedly secure strap 20 to catheter 60 and prevent any further adjustment of loop 26. Tube 44 may be displaced along catheter 60 until it abuts against or is arranged proximate to surface 59 of male connector 50 prior to crimping. In some embodiments, tube 44 comprises stainless steel. It should be appreciated that any suitable means for locking catheter 60 and strap 60 together may be used, and that this disclosure should not be limited to only the use of crimp tube 44.

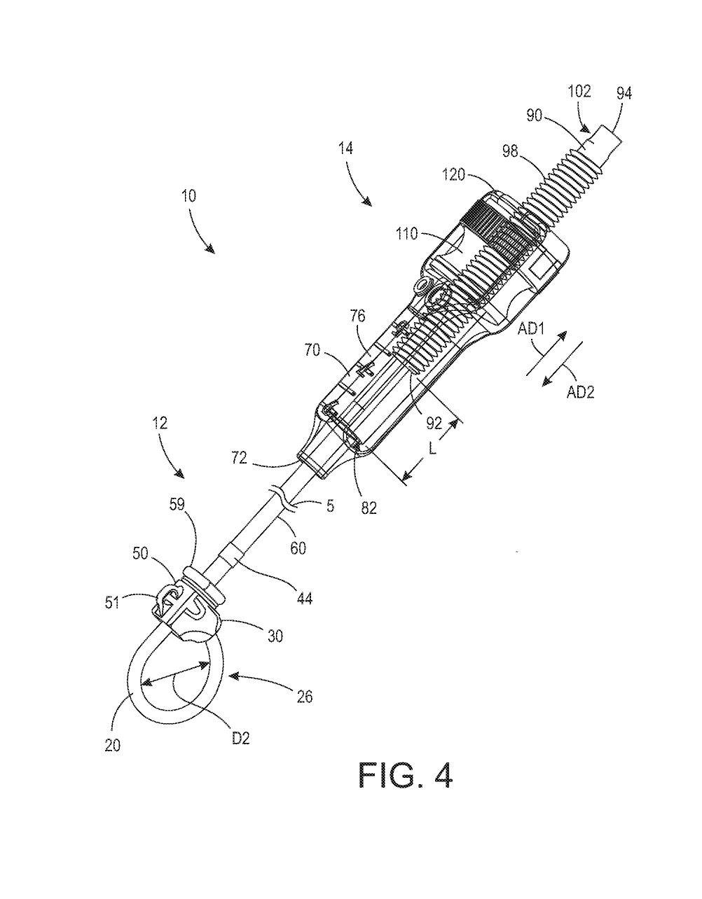

FIG. 5 is a perspective view of securing means 12. Once tube 44 is crimped thus locking catheter 60 and strap 20 together, securing means 12 may be cut, along cut line 5 for example, thus separating tensioning means 14 from securing means 12. Securing means 12, with the set desired diameter loop 26 (e.g., diameter D2) arranged around vein or vessel 2, may be left in place with minimal obstruction to the patient. Removing tensioning means 14 from securing means 12 also removes mass from flow controller 10 and may reduce pressure on vein or vessel 2.

FIG. 6 is a perspective view of housing 70. FIG. 7 is a perspective view of threaded rod 90. As previously discussed, threaded rod 90 is arranged in hole 84 of housing, with groove 104 engaged with protrusion 86. The engagement of groove 104 and protrusion 86 prevents the rotation of threaded rod 90 and thus allows the rotation of nut 110 to translate threaded rod 90 in axial direction AD1 and axial direction AD2.

Flow controller 10 can have more than one configuration for different clinical applications. For example, for an existing high flow fistula or following kidney transplantation, securing means 12 comes attached to tensioning means ready for use. After adjusting flow (i.e., setting the diameter of loop 26), tensioning means 14 may be removed. As a precaution, when creating a potential high-flow fistula, securing means 12 may be installed and left in an untightened position. In this case, end 64 of catheter 60 (arranged subcutaneously) is fitted with a connector and capped. If, subsequently, flow control is needed, the catheter end connector may be exposed and connected to a manual tensioning means with a mating connector.

Some examples of applications for which flow controller 10 may be used are AV fistula flow reduction for ischemic symptoms, AV fistula partial flow reduction following kidney transplantation, AV fistula flow reduction to prevent or treat cardiac symptoms, reduction of portal vein flow following liver transplantation, any body conduit requiring restriction or controlled flow reduction (may be in GI tract, genito-urinary system, or lungs). Flow controller 10 allows blood flow to be individually titrated to clinical symptoms for each patient. Flow controller 10 lowers stress on the vessel wall since band diameter is significantly larger (greater than 5 times) than 2-0 suture, there is no need to puncture the fistula, and there is no vessel damage from a tight suture. Flow controller 10 utilizes a TEFLON® PTFE strap, which minimizes adhesions. Flow controller 10 comes ready to use and does not require individual sizing.

It will be appreciated that various aspects of the disclosure above and other features and functions, or alternatives thereof, may be desirably combined into many other different systems or applications. Various presently unforeseen or unanticipated alternatives, modifications, variations, or improvements therein may be subsequently made by those skilled in the art which are also intended to be encompassed by the following claims.

### REFERENCE NUMERALS

2 Vein (or vessel)
5 Break line
10 Flow controller
12 Securing means
14 Tensioning means
20 Strap
22 End
24 End
26 Loop
30 Female connector
32 Radially inward facing surface
34 Notch (notches)
36 Groove
38 Hole
40 Bushing
42 Suture
44 Tube
50 Male connector
51 Attachment
52 Radially outward facing surface
54 Protrusion (protrusions)
56 Groove
58 Through-bore
59 Surface
60 Catheter
62 End
64 End
70 Housing
72 End
74 End
76 Measuring surface
78A Fork portion
78B Fork portion
80 Hole
82 Surface
84 Hole
86 Protrusion
90 Threaded rod
92 End
94 End
96 Radially outward facing surface
98 Threading
100 Through-bore
102 Crimp portion
104 Groove
106 Washer
108 Washer
110 Nut
112 Grip
114 Hole
120 Housing cap
122 Hole
124A Clip portion
124B Clip portion
D1 Diameter
D2 Diameter
L Length
AD1 Axial direction
AD2 Axial direction
CD1 Circumferential direction
CD2 Circumferential direction

What is claimed is:

1. A flow controller arranged to provide controlled constriction of a hollow body conduit, comprising:
a catheter including a first end and a second end;
a strap slidably arranged within the catheter, the strap including a third end and a fourth end, the third end arranged to be connected to the catheter to form a loop; and,
a tensioning means connected to the second end and the fourth end, wherein the tensioning means is operatively arranged to axially displace the strap relative to the catheter to increase and decrease a diameter of the loop.

2. The flow controller as recited in claim 1, further comprising a first connector fixedly secured to the first end, the third end being removably connectable to the first connector.

3. The flow controller as recited in claim 2, further comprising a second connector fixedly secured to the third end, the second connector being removably connectable to the first connector.

4. The flow controller as recited in claim 3, wherein:
the first connector comprises a first annular groove;
the second connector comprises a second annular groove; and,
a suture is arranged in the first and second annular grooves to fixedly secure the second connector to the first connector.

5. The flow controller as recited in claim 3, wherein:
one of the first connector and second connector comprises an alignment protrusion; and,
the other of the first connector and the second connector comprises an alignment notch; and,
when the second connector is connected to the first connector, the alignment notch engages the alignment protrusion to properly align the first and second connectors.

6. The flow controller as recited in claim 1, further comprising a tube operatively arranged on the catheter, wherein the tube may be crimped to lock the strap and catheter together.

7. The flow controller as recited in claim 2, wherein the first connector further comprises an attachment operatively arranged for connection of the first connector to surrounding tissue.

8. The flow controller as recited in claim 1, wherein the tensioning means comprises:
a housing connected to the second end; and,
a rod slidably arranged in the housing, wherein the rod is connected to the fourth end and is displaced relative to the housing to increase and decrease the diameter of the loop.

9. The flow controller as recited in claim 8, further comprising a nut arranged at least partially within the housing and threadably engaged with the rod, wherein displacement of the nut in a first circumferential direction displaces the rod in a first axial direction.

10. The flow controller as recited in claim 8, wherein the housing comprises a first hole and a second hole, the rod being engaged with the second hole.

11. The flow controller as recited in claim 10, wherein:
the housing further comprises a protrusion arranged in the second hole; and,
the rod comprises a groove, the groove operatively arranged to engage the protrusion to prevent rotation of the rod relative to the housing.

12. The flow controller as recited in claim 10, wherein the rod further comprises a through-bore and the fourth end is fixedly secured therein.

13. A flow controller arranged to provide controlled constriction of a hollow body conduit, comprising:
a catheter including a first end and a second end;
a strap slidably arranged within the catheter, the strap including a third end and a fourth end, the third end operatively arranged to be removably connected to the catheter to form a loop, wherein the loop is arranged around the hollow body conduit; and,
a tensioning means, including:
a housing connected to the second end; and,
a threaded rod slidably arranged in the housing, wherein the threaded rod is connected to the fourth end and is axially displaced relative to the housing to increase and decrease a diameter of the loop.

14. The flow controller as recited in claim 13, further comprising a first connector fixedly secured to the first end, the third end being removably connectable to the first connector.

15. The flow controller as recited in claim 14, further comprising a second connector fixedly secured to the third end, the second connector being removably connectable to the first connector.

16. The flow controller as recited in claim 13, wherein:
one of the first connector and second connector comprises an alignment protrusion; and,
the other of the first connector and the second connector comprises an alignment notch; and,
when the second connector is connected to the first connector, the alignment notch engages the alignment protrusion to properly align the first and second connectors.

17. The flow controller as recited in claim 13, further comprising a tube operatively arranged on the catheter, wherein the tube may be crimped to lock the strap and catheter together.

18. The flow controller as recited in claim 13, further comprising a nut arranged at least partially within the housing and threadably engaged with the threaded rod, wherein displacement of the nut in a first circumferential direction displaces the rod in a first axial direction.

19. The flow controller as recited in claim 13, wherein:
the housing comprises a first hole in which the second end is fixedly secured and a second hole having a protrusion arranged therein; and,
the rod is at least partially arranged within the second hole and comprises a groove, the groove operatively arranged to engage the protrusion to prevent rotation of the rod relative to the housing.

20. The flow controller as recited in claim 13, wherein the rod further comprises a through-bore and the fourth end is fixedly secured therein.
